(19) United States
(12) Patent Application Publication
(10) Pub. No.: US 2018/0320311 A1
(43) Pub. Date: Nov. 8, 2018

(54) DYE DISCHARGE REAGENT FOR INKJET COMPOSITIONS

(71) Applicant: Kornit Digital Ltd., Rosh HaAyin (IL)

(72) Inventor: Jacob MOZEL, Kfar-Saba (IL)

(21) Appl. No.: 15/771,096

(22) PCT Filed: Nov. 9, 2016
(86) PCT No.: PCT/IL2016/051208

§ 371 Date: Apr. 27, 2018

Related U.S. Application Data
(60) Provisional application No. 62/252,648, filed on Nov. 9, 2015.

(51) Int. Cl. D06P 5/15; D06P 5/30

(52) U.S. Cl. CPC D06P 5/155; C07C 313/04; D06P 5/30

(57) ABSTRACT

A process of in situ forming a dye discharge reagent on at least a portion of surface of a dyed substrate, effected by applying two or more compositions comprising reactants on the substrate, such that the reactants undergo a chemical reaction on the substrate to form the reagent is provided herein, as well as a method for dye discharging a dyed substrate using the process of in situ forming a dye discharge reagent, and a dye discharge composition comprising said dye discharge reagent. In some embodiments of the invention, the reactants are thiourea and a peroxide, and the dye discharge reagent is formamidine sulfinic acid (FSA).

### FIELD AND BACKGROUND OF THE INVENTION

The present invention, in some embodiments thereof, relates to textile printing and, more particularly, but not exclusively, to dye discharge compositions and use thereof in inkjet printing.

Dye discharge printing, also known as extract printing, is a method of forming a design onto a dyed substrate, typically a fabric, by printing thereon a color-destroying (dye discharging) agent, such as chlorine or hydrosulfite, to bleach out a light pattern on the darker colored substrate. In color-discharge printing, a dye or pigment, which is impervious to the discharging agent, may be combined with the reducing (discharge) agent so as to color the discharged part of the substrate thereby imparting a colored design instead of dye-free areas on the substrate.

Discharge printing has the ability to make bright, opaque colors on dark fabrics, while keeping the substrate's surface smooth and allowing the finished fabric to exhibit a soft hand-feel owing to the fact that substance is withdrawn rather than added to the fabric in the printing process.

Newly developed discharge ink systems involve highly reactive chemicals that are typically derived from stable compounds by decomposition at high temperature. This advancement opened the door to discharge printing for the standard screen (stencil) printing techniques.

Successful light-on-dark printing with standard inks relies on a white background silhouette or a white underbase layer, increased pigment loads, fillers and other additives to block out the color of the substrate. Discharge inks modify the garment color by removing the substrate's original color and optionally replacing it with the new ink color.

One of the most promising technologies for printing high quality color designs and images, particularly in small batches of varying contents (short runs of variable data), on a wide variety of types and shapes of substrates, such as textile surfaces, is inkjet printing. Inkjet printing is a wide-spread technique in which a stream of a specific liquid ink composition is ejected as droplets from a cluster of minute nozzles (printheads) in response to electrical signals generated by a microprocessor to record characters and patterns on the surface of a printed substrate without making direct contact between the ink application apparatus and the surface of the subject (non-impact printing). A typical inkjet printing system includes methods and apparatus in which electric signals are converted to mechanical signals for a continuous or on-demand jetting of an ink composition which is continuously supplied and stored in a nozzle head portion, to thereby record characters, symbols and patterns on the surface of a substrate.

To ensure high quality images by inkjet, all inkjet compositions should be characterized by free passage through the nozzles, minimal bleeding, paddling and/or smearing, uniform printing on the surface of the substrate, wash-fastness, simple system cleaning and other chemical and physical characteristics. Thus, inkjet ink compositions characterized, for example, by extended chemical and physical stability, suitable viscosity, solubility, volatility, surface tension, compatibility with other components of the printing system and, in cases of continuous flow inkjet printing, electrical resistance, and further by being applied using suitable devices, techniques and processes, are continuously sought for.

Dye discharge ink compositions are typically suitable for screen printing, and are less suitable for inkjet settings, due to, for example, incompatible high viscosity, incompatible particulate matter, corrosive nature, and chemical and physical instability that ultimately lead to decomposition and sedimentation and hence short shelf life of the ink composition.

Texcharge® TC by Sericol® is a water-based discharge ink system, designed to provide maximum opacity and impact when printed on reactive dyed cottons. This system is designed for the silk or screen printing technologies, namely the compositions are thick and implemented as pastes, allowing them to pass through the mesh under pressure while limiting spreading and feathering into and under the masked areas.

Mimaki Engineering Company (Japan) is offering GP-1810D® and GP-604® Series of printing machines which are said to be compatible for discharge printing using a discharge liquid.

WO 2014/076705 and WO 2015/025310, by the present assignee, which are incorporated by reference as if fully set forth herein, teaches dye discharge compositions exhibiting extended shelf life and/or pot life and high suitability for inkjet printing on dyed substrates. The disclosed compositions comprise a reducing agent such as, for example, a sulfur-based reducing agent, and a chelating agent, such as sodium salt of a polyamino carboxylic acid chelating agent. The compositions disclosed in WO 2014/076705 and WO 2015/025310 can be used in combination with a translucent colored composition and an optional opaque underbase composition, for forming an image on a dyed substrate.

Formamidine sulfinic acid, also known as formamidinesulfinic acid (FSA) or thiourea dioxide (TUDO), is an oxidation product of thiourea and a well-known reducing agent used in biology, paper, wool, textile and other industrial purposes. Due to thermal decomposition that releases SO₃, it can be used as a dye discharge agent for dyed cotton fabrics, and more commonly for dye discharge of cellulosic fibers dyed by sulfur-based dyes (sulfur dyes). FSA is stable both in solid form and in cold aqueous solution, and has a slight acidic dye discharge reaction. FSA acquires full reducing power when heated in aqueous solution to about 100° C. U.S. Pat. Nos. 2,150,921 and 2,783,272 disclose that FSA is synthesized from thiourea and hydrogen peroxide, essentially by following reaction:

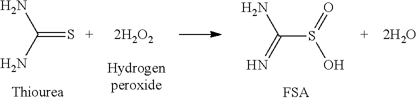

In the textile industry, FSA is used for dye discharge in place of the traditional zinc or sodium formaldehyde sulfoxylate reducing agent, where it is used to discharge dyed garments in screen printing settings as a thin grounded powder that is mixed into water-based pastes, and applied through suitable mesh. When the garment is dried in a conveyer drier, FSA reacts with the dyes in the fabric. Krug, P. [Journal of the Society of Dyers and Colourists, 1953, 69(13), pp. 606-611] describes the uses of FSA in textile printing, while reporting the following aqueous thermal decomposition of FSA to urea and sulfoxylic acid:

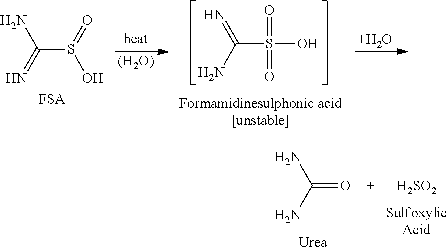

Attempts to use FSA in inkjet ink formulations for digital prints is impractical due to the poor solubility of FSA in aqueous solutions. The aqueous solubility of FSA is about 2 grams per 100 ml of water at 20° C. FSA is more soluble in hot water, but precipitates rapidly when the solution is cooled. FSA is more soluble in basic solutions, but decomposes rapidly therein. FSA is also poorly soluble in polar organic solvents. Hence, its use for preparing water soluble low viscosity inks is rather limited, and rendered impractical for inkjet applications.

U.S. Pat. No. 5,149,871 describes a procedure for synthesizing water-soluble derivatives of FSA that includes reacting FSA with a series of amino acids such as glycine, β-alanine, 4-amino N-butyric acid and others to afford a soluble compound when neutralized by sodium, potassium or calcium acetate salts.

### SUMMARY OF THE INVENTION

Embodiments of the present invention relate to a process of in situ formation of reagents that are useful in inkjet printing, such as dye discharge reagent, directly on the surface of the substrate. This in situ formation is afforded by applying compositions that contain reactants that form the reagent, using separate applicators, onto the substrate, thereby combining the reactants in a concerted chemical reaction, which becomes part of the printing process. Since the reagent is formed from two stable reactants that are kept apart until used in a printing process, the process is advantageous chemically, mechanically, economically and environmentally.

Embodiments of the present invention further relate to the use of the herein disclosed dye discharge composition, optionally in combination with a colored ink composition, for printing an image on a dyed substrate.

According to an aspect of some embodiments of the present invention, there is provided a process of in situ forming a dye discharge reagent on at least a portion of a dyed substrate, which includes contacting a first composition and a second composition on the portion, wherein the first composition includes a first reactant and the second composition includes a second reactant, the first reactant and the second reactant undergo a chemical reaction in situ on the substrate, thereby forming the dye discharge reagent.

According to an aspect of some embodiments of the present invention, there is provided a method of discharging a dye in a dyed substrate, which includes contacting at least a portion of the substrate with a first composition and a second composition, wherein the first composition includes a first reactant and the second composition includes a second reactant, the first reactant and the second reactant undergo a chemical reaction to form a dye discharge reagent in situ on the substrate, thereby discharging the dye in the substrate with the dye discharge reagent.

In some embodiments, contacting at least a portion of the substrate with the first and the second composition is afforded by separately applying the first composition and the second composition on the portion.

In some embodiments, the first reactant and the second reactant are applied at a stoichiometric ratio.

In some embodiments, applying the first composition and the second composition is effected by inkjet printing at least one of the first and second compositions.

In some embodiments, each of the first composition and the second composition is individually applied on the portion of the substrate by a separate inkjet printhead.

In some embodiments, the inkjet printhead is an ink circulation printhead.

In some embodiments, the applying the first composition and the second composition is effected concertedly, concomitantly or sequentially.

In some embodiments, the chemical reaction is initiated upon the contacting.

In some embodiments, the process and/or the method presented herein further includes heating at least the portion of the substrate subsequent to contacting the first and second composition with the substrate.

In some embodiments, heating is effected at a temperature that ranges from 100° C. to 160° C.

In some embodiments, the process and/or the method presented herein further includes irradiating at least the portion of the substrate subsequent to the contacting.

In some embodiments of the process and/or the method presented herein, the first reactant is thiourea and the first composition is an aqueous solution of the thiourea.

In some embodiments of the process and/or the method presented herein, the second reactant is a peroxide and the second composition is an aqueous solution of the peroxide.

In some embodiments of the process and/or the method presented herein, the dye discharge reagent formed in situ is formamidine sulfinic acid (FSA).

According to an aspect of some embodiments of the present invention, there is provided an inkjet dye discharge composition that includes a first composition and a second composition, wherein the first composition is an aqueous solution of thiourea and the second composition is an aqueous solution of a peroxide.

In some embodiments of the process and/or the method and/or the composition presented herein, the concentration of the thiourea in the first composition ranges from 5-25%.

In some embodiments of the process and/or the method and/or the composition presented herein, the peroxide is selected from the group consisting of hydrogen peroxide, peroxymonosulfuric acid, a peroxomonosulfate salt, a peroxydisulfate salt, an organic peroxy acid, an organic peroxide, an organic hydroperoxide, a perester and a transition metal peroxide.

In some embodiments of the process and/or the method and/or the composition presented herein, the concentration of the peroxide in the second composition ranges from 5-50%.

According to an aspect of some embodiments of the present invention, there is provided a dyed textile substrate, wherein at least a portion of the substrate is dyed discharged using the dye discharge reagent that is formed in situ on the substrate, as presented herein.

According to an aspect of some embodiments of the present invention, there is provided a dyed textile substrate, wherein at least a portion of the substrate is dyed discharged using any of the methods presented herein.

According to an aspect of some embodiments of the present invention, there is provided a dyed textile substrate, wherein at least a portion of the substrate is dyed discharged by any of the inkjet dye discharge compositions presented herein.

According to an aspect of some embodiments of the present invention, there is provided a kit that includes a packaging material and the inkjet dye discharge composition of any one of claims 16-19 packaged therein.

In some embodiments of the kit presented herein, the kit is identified for use in discharging a dye in a dyed textile substrate.

In some embodiments of the kit presented herein, the kit is identified for use in a method of inkjet printing an image on at least a portion of a dyed textile substrate.

In some embodiments of the kit presented herein, the first composition and the second composition are packaged individually within the kit.

In some embodiments of the kit presented herein, the kit further includes instructions that read:

load the first composition to a first printhead;

load the second composition to a second printhead; and

contact the first composition with the second composition by inkjet printing the first composition and the second composition on a substrate.

In some embodiments of the kit presented herein, the kit further includes a translucent colored composition packaged individually within the kit.

In some embodiments of the kit presented herein, the kit further includes an opaque underbase composition packaged individually within the kit.

According to an aspect of some embodiments of the present invention, there is provided a process of discharging a dye from at least a portion of a dyed textile substrate, the process includes digitally applying, by means of inkjet printing onto the portion, any of the inkjet dye discharge compositions presented herein.

In some embodiments, the process further includes, subsequent to the applying the inkjet dye discharge compositions presented herein, heating the substrate.

According to an aspect of some embodiments of the present invention, there is provided a process of inkjet printing an image on at least a portion of a dyed textile substrate, that includes digitally applying onto at least a portion of a substrate any of the inkjet dye discharge compositions presented herein, and digitally applying substantially over the portion a translucent colored composition, thereby forming the image.

In some embodiments, the process further includes, prior to applying the translucent colored composition, digitally applying an opaque underbase composition substantially over the portion.

In some embodiments of the process, applying the dye discharge composition and applying the translucent colored composition, and/or applying the dye discharge composition and applying the opaque underbase composition, if present, is effected substantially concurrently.

In some embodiments of the process, the process further includes, subsequent to the applying, heating the substrate.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a photograph of an exemplary Discharge/Devoré Selection Chart, which was formed on a sample of a brown-dyed cotton fabric sample on which a dye-discharge composition, according to some embodiments of the present invention, was applied, showing the various dye-discharge and/or fabric burnout (devoré) levels resulting for the application of various amounts of the two components comprising the dye discharge composition.

### DESCRIPTION OF SPECIFIC EMBODIMENTS OF THE INVENTION

The present invention, in some embodiments thereof, relates to textile printing and, more particularly, but not exclusively, to dye discharge compositions and use thereof in inkjet printing.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Dye discharge techniques can produce soft and durable designs, however these currently known dye discharge techniques for textile are limited in the spectrum of dye discharge agents and compositions, as well as the printing machinery and techniques which can employ such agents, which are typically incompatible with inkjet techniques.

As discussed hereinabove, formamidinesulfinic acid (FSA) is a well-known dye discharge agent that is widely used in screen printing techniques, but is found impractical for inkjet techniques due to its low solubility in root temperature aqueous solutions, and its tendency to decompose in hot aqueous solutions, rendering FSA useless as discharge reagent in low viscosity inkjet ink formulations.

While contemplating the use of FSA as a dye discharge agent for the field of inkjet printing on dyed substrates, the present inventor has envisioned a dye discharge composition wherein FSA is produced in situ on the surface of the printed substrate by separately jetting inkjet-suitable reagents that form FSA when coming in contact with one-another. The present inventor has envisioned FSA-forming reagents formulated in separate inkjet-suitable formulations, concertedly jetted at pre-determined relative amounts (stoichiometric ratio) and reacting on the substrate to form FSA, which is suitable for dye discharge printing. It has further been contemplated to use one of the FSA-forming reagents in excess with respect to the other reagent so as to afford an aesthetic effect in the fabric, such as a burnout effect, known in the industry as devoré, which is used for fashion products.

While reducing the present invention to practice, the inventor has successfully produced FSA in situ on the fabric while jetting thiourea solution from one printhead and hydrogen peroxide solution from another printhead. As only very small amounts are jetted of each component, no external cooling was required to attenuate the exothermic reaction or an undesired burnout of the fabric.

It was further found that when higher than stoichiometric ratio (relative amounts) of the oxidizer is jetted, cellulosic substrates exhibit a controllable burnout (devoré) effect. Therefore by controlling the ratio of thiourea and peroxide on the substrate, two phenomena of fabric dye discharge and fabric burnout were achieved. Hence, the object of present invention is also to provide a procedure to generate FSA in situ on garments or textile, and using the exothermic reaction for more desired features (devoré) while printing on garments or textile.

### Process for In Situ Synthesis of a Reagent for Inkjet:

The present inventors have contemplated a general methodology of in situ synthesis of chemical reagents which are useful in the field of printing, on at least a portion of a surface of a substrate. The inventors have contemplated taking advantage of inkjet equipment and setting to controllability deliver several separated liquid compositions to specific and pre-determined areas of the surface of any substrate, and have these compositions be contacted with one another only on the surface of the substrate such that desired reagents are formed as a result of a chemical reaction between various reactants delivered by the separated liquid compositions. The chemical reaction can take place spontaneously or upon applying heat or radiation to the mixed compositions on the surface of the substrate.

The general methodology of in situ synthesis of a reagent on a substrate, or at least a portion of a surface thereof, is particularly advantageous for reagents that are useful for printing but are incompatible with inkjet printing processes due to any reason, including, but not limited to solubility, corrosiveness/corrosivity, stability, compatibility and the like. Specifically, a reagent that is useful for printing can be a substrate dye discharge reagent, a substrate modifying reagent, a colorant, an ink viscosity modifying reagent and the likes. Such a reagent may be insoluble at the required concentration in the carrier/medium of the ink composition, and/or chemically instable to be part of an ink composition that is used for an extended period of time, and/or reactive with other components in the ink composition, and/or reactive with parts of the printing machinery (printhead tubing, nozzles, circuitry and the like), or expensive compared to the starting materials of its synthesis. As discussed hereinabove, an example of such a reagent is the dye discharge reagent FSA.

Thus, according to an aspect of some embodiments of the present invention, there is provided a process of in situ forming a dye discharge reagent on at least a portion of a dyed textile substrate; the process is effected by contacting a first composition (also referred to as a first delivery composition) and a second composition (also referred to as a second delivery composition) on the substrate (e.g., a surface thereof), wherein the first composition includes a first reactant and the second composition includes a second reactant, such that the first reactant and the second reactant undergo a chemical reaction in situ on that portion of the substrate, thereby forming the dye discharge reagent.

The description below is using the example of two compositions, each carrying a single reactant only for the same of simplicity. Hence, it is noted that the invention is not limited to a chemical reaction between two reactant each carried by a separate composition, and that embodiments of the present invention encompass a chemical reaction involving 2, 3, 4, 5, 6 and more reactants. It is also noted that some of the reactants can be delivered to the substrate in a common delivery composition, while some may be delivered in separate delivery compositions; hence the invention is not limited to the use of two separate delivery compositions to form the reaction mixture, and that embodiments of the present invention encompass a chemical mixture that is afforded by combining 2, 3, 4, 5, 6 and more compositions.

According to some embodiments of the present invention, contacting the first and second composition forms a combined composition which can be seen as a reaction mixture, in which the first and second reactant react with one another to form the reagent in situ. The combined composition is the reaction mixture and also the composition that contains the in situ synthesized reagent after the chemical reaction takes place. For example, according to some embodiments of the present invention, the in situ synthesized reagent is a dye discharge reagent, and the combined delivery compositions may contain ingredients other than the reactants that react to form the dye discharge reagent. In such embodiments, the combined composition, which is also the reaction mixture in which the chemical reaction takes place, becomes a dye discharge composition. According to some embodiments of the present invention, the in situ synthesized reagent is a colorant, and the combined composition becomes a colored ink composition once the chemical reaction takes place.

According to some embodiments of the present invention, the chemical reaction initiates upon contacting the first and second compositions, as in the case of a spontaneous initiation of a reaction between two reactants. According to some embodiments of the present invention, the chemical reaction initiates upon contacting the first and second compositions and generating at least one additional condition to initiate the chemical reaction, such as applying heat to at least that portion of the substrate where the two compositions come in contact, and/or applying radiation (irradiating) the reaction mixture at an energy level suitable for initiating the chemical reaction between the reactants. According to some embodiments of the present invention, the chemical reaction initiates upon contacting the first and second compositions and a third reactant that is already present on the substrate prior to contacting the first and/or second compositions with the substrate.

The term “chemical reaction”, as used herein, refers to a process that takes place in a reaction mixture that includes reactants, and leads to the formation of a chemical entity that was not present in the reaction mixture prior to the process, or to the transformation of at least one of the reactants to another chemical entity. In general, a chemical reaction involves the positions of electrons and the forming and breaking of chemical bonds between atoms. According to some embodiments of the present invention, a chemical reaction involves the breaking and/or formation of covalent bonds between atoms which formed a part of at least one reactant in the reaction mixture. According to such embodiments, the formation of a FSA from thiourea and hydrogen peroxide is a chemical reaction, while the protonation of acetate to form acetic acid, or deprotonation of ammonium to ammonia do not constitute a chemical reaction.

### Method for Dye Discharging a Dyed Substrate:

In accordance to the above, there is provided a method of discharging a dye in a dyed substrate, or at least a portion thereof, which is effected by contacting at least a portion of the substrate with a first composition and a second composition, wherein the first composition includes a first reactant and the second composition includes a second reactant, the first reactant and the second reactant undergo a chemical reaction to form a dye discharge reagent in situ on the substrate, thereby discharging the dye in the substrate with the dye discharge reagent.

As used herein, the phrase “dyed substrate”, which is also referred to herein interchangeably as a “dyed surface”, a “colored substrate”, a “colored surface”, and a “darkly-colored surface”, refers to a substrate, or a surface of a substrate, having any color which is not white (non-white), such as for example, a yellow substrate, a gray substrate, a red substrate, a black substrate and the likes.

According to some embodiments of the present invention, the lightness of a darkly-colored substrate or of its surface has a color which is attributed an L* (lightness) value of 50 or less and any a* and b* values on the L*a*b* scale, as discussed herein. As used herein, the term “L*a*b*” or “Lab*” refers to the CIE L*a*b* (International Commission on Illumination or Commission Internationale d'Eclairage, CIE) color model. Used interchangeably herein and throughout, CIE L*a*b*, L*a*b* or Lab is the most complete color model used conventionally to describe all the colors and shades which are typically visible to a normal human eye. The three parameters in the model define a particular color, whereas the lightness of the color is represented by the parameter L*, wherein L*=0 corresponds to black and L*=100 corresponds to white. The value between true magenta and true green is represented by the parameter a*, wherein a negative value indicates green and a positive value indicates magenta. The value between true yellow and true blue is represented by the parameter b*, wherein a negative value indicates blue and a positive value indicates yellow.

According to some embodiments of the present invention, the substrate is a textile substrate, such as a woven or non-woven fabric, a cloth, a garment and/or a piece of clothing.

Since the first and second reactants in the first and second compositions may react with one another prematurely, the first and second compositions are kept separated until they are applied on the substrate. According to some embodiments, contact between the first and second reactants takes place when the first and second compositions come in contact upon applying the compositions on the substrate, preferably at predetermined areas (portion) of the surface of the substrate.

In inkjet printing settings, according to some embodiments of the present invention, at least one of the first and second compositions are applied on the substrate using an inkjet printhead. In some embodiments, both the first and second compositions are applied individually on the substrate using a different inkjet printhead for each of the compositions. The printhead can be any printhead known in the art, as long as it is suitable for carrying and applying (jetting) the delivery composition contained therein. For example, in some embodiments, the printhead is preferably a circulation printhead, which is advantageous when applying a delivery composition that contains a reactant which requires circulation is order to be printed on the substrate. It is noted that reactants such as peroxides may have a tendency to form bubbles (of O₂) which may clog the printhead unless the solution the peroxide is mixed/dissolved in is circulated.

According to some embodiments, the compositions comprising the reactants, e.g., the first and second compositions, are applied on the substrate such that the two compositions can mix into a reaction mixture, thereby bringing the reactants, which were kept separately up to this point, into contact. The compositions may be applied to the substrate at different time points as long as the two compositions can still mix, at least to some extent, and the reactant can come in contact. According to some embodiments, the first and second compositions are applied on the substrate concurrently (simultaneously), concertedly, concomitantly or sequentially (successively) at any order. The time period between applying any one of the first or second compositions, and applying the other composition, may be 0 (simultaneously), less than 0.01 seconds, less than 0.1 seconds, less than 1 second, less than 2 seconds, less than 3 seconds, less than 4 seconds, less than 5 seconds, less than 6 seconds, less than 7 seconds, less than 8 seconds, less than 9 seconds, or less than 10 seconds. It is noted that other longer time periods are also contemplated within the scope of some embodiments of the present invention.

According to some embodiments of the present invention, the chemical reaction between the reactants in the combined first and second compositions (the reaction mixture) is initiated upon contacting the first and second compositions. In some embodiments, the chemical reaction initiates spontaneously, requiring no input of free energy to go forward.

In some embodiments, the chemical reaction requires input of free energy to initiate and go forward. In such cases the reaction initiates upon contacting the first and second compositions and further upon applying energy thereto in the form of heat, radiation and the like. Heating the reaction mixture can be effected by heating at least the portion of the substrate where the reaction mixture is formed. Irradiating the reaction mixture can be effected by passing the substrate having the mixture thereon under a light source such as IR or UV. Heating the reaction mixture can be effected by convection, hot air, infrared irradiation, heat coils and the like. The heat is applied so as to raise the temperature of the reaction mixture to a temperature that is sufficient to initiate the chemical reaction between the reactants. For example, the reaction mixture can be heated to more than 30° C., more than 40° C., more than 50° C., more than 60° C., more than 70° C., more than 80° C., more than 90° C., more than 100° C., more than 110° C., more than 120° C., more than 130° C., more than 140° C., or more than 150° C. According to some embodiments, heating is effected at a temperature that ranges from 100° C. to 160° C.

Heating the substrate on which the reagent is formed in situ is also performed in cases the reagent requires energy to confer its activity on the substrate. For example, an aqueous dye discharge composition comprising FSA confers its dye discharging activity when heated to a temperature of about 100° C. or higher. Hence, in some embodiments of a method of discharging a dye in a dyed substrate, at least the portion of the surface on which the dye discharge reagent is formed in situ, is heated to about 100° C. or higher in order to initiate the dye discharge reaction.

In embodiments where the chemical reaction is based on a particular stoichiometry of reactants and products, the first and second reactants are applied on the substrate in amounts that match the stoichiometry of the chemical reaction, or in other words, the amounts of the reactants match the stoichiometric ratio of the reaction. The stoichiometric ratio is the relative amount of one reactant to completely react with the other reactant in a chemical reaction, or the stoichiometric amounts that would result in no excess reactants when the reaction takes place. For example, in a chemical reaction that requires one molecule of the first reactant and one molecule of the second reactant to produce one molecule of the product, the stoichiometric ratio is 1:1 and thus the amounts of the first reactant and the second reactant are essentially equal. In a chemical reaction that requires one molecule of the first reactant and two molecules of the second reactant to produce one molecule of the product, the stoichiometric ratio is 1:2. In some embodiments, the amounts of the reactants deviates from the stoichiometric ratio, namely at least one of the reactants is applied on the substrate in excess, relative to the other reactant(s). In these embodiments the motivation to use an excess of a reactant may stem from a particular property of that reactant which is beneficial for the printing process (for example, acidity or pH, ionic strength) or that a property of the other reactant is undesired and the chemical reaction is required to consume the entire amount of that other reactant (the reactant that is used below its stoichiometric amount). In some embodiments the reactant that is used above its stoichiometric amount (major reactant) is not harmful to the printing process or the finished product, and/or can be easily be rid of, and reactant that is used below its stoichiometric amount (the minor reactant) is more expensive and/or less stable and/or harder to be rid of, and the chemical reaction is designed to be driven to completion while consuming all the minor reactant.

In some embodiments, the amounts of the reactants in the reaction mixture are pre-determined based on their concentration in the delivery compositions, the volume of the delivery compositions applied on the substrate (amount per area; drop size and density etc.), and/or the number of passes by which each delivery composition is applied on the substrate. The total amount of the product of the chemical reaction, according to embodiment of the present invention, is determined according to the desired effect which the reagent is intended to afford on the substrate. For example, if the reagent is a colorant and the desired effect is coloring the substrate, the amount of the product resulting from the chemical reaction is the amount that would confer the desired color intensity to the substrate. If the reagents is a dye discharge reagent, the amount of the product resulting from the chemical reaction is the amount that would confer the desired dye discharge to the dyed substrate. In some embodiments, the reagent is capable of various effects as different amounts, as in the case of a dye discharge reagent that can mildly discharge the dyed substrate at low amounts, bleach the substrate at relatively high amounts, and possibly burnout the substrate at higher amounts.

According to some embodiments of the present invention, at least one of the compositions containing the reactants is an aqueous solution of the reactant, namely the main carrier of the composition is water. In some embodiments, both the first and the second compositions are aqueous solutions of the first and second reactants, respectively.

### Dye Discharge Reagent and Inkjet Dye Discharge Composition:

In some embodiments of the present invention, the reagent is a dye discharge reagent. According to some embodiments of the present invention, the dye discharge reagent is a sulfur-based reducing agent, such as FSA. Like other reducing agents, sulfur-based reducing agents are capable of causing a dye to lose its light interaction properties such that it appears to change color or become colorless. Such processes are also known as bleaching in the broad sense of the term. The color of a substance (a substrate or a surface thereof) is typically a result of light interacting with compounds called chromophores. Chemical bleaching may be effected by oxidation or reduction of the chromophores such that these compounds no longer interact with light in the same manner as before the bleaching reaction. Hence, in the context of embodiments of the present invention, dye discharge and bleaching are terms that are used to refer to the result of loss of color or elimination of the ability of the chromophore to absorb and/or reflect visible light, regardless of the effector being an oxidizing or a reducing agent.

Without being bound by any particular theory, a dye discharge reaction, or bleaching, is most effective for a dye that has a chemical structure based on long pi-conjugated systems which include reduction-susceptible double bonds, such as found in many sulfur and azo-based dyes.

As discussed hereinabove, one of the more useful dye discharge reagents in the printing industry is formamidine sulfinic acid (FSA). This reagent is known for being safe and environmentally sound, whereas the major limitation preventing is use in inkjet settings is its low solubility in water or other inkjet-suitable carriers that are compatible with the machinery and the substrates (e.g., textile). As discussed hereinabove, FSA can be synthesized from a peroxide and thiourea, which react spontaneously upon contacting one another in aqueous media, whereas heat activates the reducing power of FSA, rendering FSA an effective dye discharge reagent prepared in situ, according to some embodiments of the present invention.

In some embodiments wherein FSA is the reagent for in situ generation, the first reactant is thiourea and the first composition is an aqueous solution of thiourea. Accordingly, the second reactant is a peroxide and the second composition is an aqueous solution of that peroxide.

The thiourea and the peroxide react with one another at a 1:2 respective stoichiometric ratio, hence in some embodiments that first reactant is thiourea and the second reactant is a peroxide, and the two are applied to the substrate at a stoichiometric ratio (1:2). In some embodiments, the peroxide is applied in an excess, and in some embodiments the thiourea is applied to the substrate in excess. When an acidic peroxide is applied to the substrate in excess, the portion of the substrate applied therewith is acidic, which can be useful is some embodiments, as discussed hereinbelow.

It is noted that the reaction between peroxide and thiourea is exothermic. In some embodiments of the present invention, the amount of FSA produced in situ is sufficiently small so as not to release heat at levels that may affect the printing process or the substrate. In some embodiments, the final amount of FSA per area, that is produced in situ, as presented herein, is determined based in the dyed substrate, the desired level of discharge and other factors such as desired devoré effect and the like. According to some embodiments of the present invention, the estimated final amount of FSA per area ranges from 5 mg to square inch to 6 mg/in², which are obtained from reacting in situ about 3.5 mg/in² to 4.5 mg/in² thiourea and about 3.1 mg/in² to 3.7 mg/in² hydrogen peroxide, whereas excess of peroxide can afford fabric burnout (devoré).

In terms of mechanical printing setting of the first and second compositions, these may be printed, according to some embodiments of the present invention, at a resolution of about 545×540 dpi, setting the drop size to 80-90 ng. It is to be understood that other printing resolutions and drop sizes are contemplated within the scope of the present invention.

### Thiourea:

One of the reactants that affords the dye discharge reagent is a thioamide of the family of thioureas. In the context of some embodiments of the present invention, the term “thiourea” refers to a family of compounds represented by the general formula I:

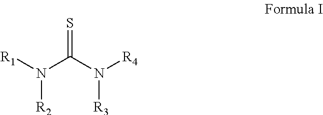

wherein each of R₁-R₄ is independently hydrogen or a low alkyl (an alkyl having from 1-4 carbon atoms). Exemplary members of the family include, without limitation, 1-methylthiourea, 1,3-dimethylthiourea and 1,1,3,3-tetramethylthiourea.

According to some embodiments of the present invention, the thioamide is thiourea (SC(NH₂)₂), which is the simplest member of the family wherein each of R₁-R₄ is hydrogen.

According to some embodiments of the present invention, the concentration of the thiourea in the first composition ranges from 5-25% by weight of the first composition (0.065M-0.325M). In some embodiments, the concentration of the thiourea in the first composition is 10-15% by weight of the first composition, or 0.13M-0.2M. In some embodiments, the concentration of the thiourea in the first composition is determined according to the concentration of the peroxide in the second composition, where in some embodiments the concentration is such that the first reactant (thiourea) is in excess with respect to the second reactant (peroxide) when the first composition and the second compositions are combined, in some embodiments the concentration is half of the concentration of the peroxide such that the first reactant (thiourea) is in a stoichiometric ratio with respect to the second reactant (peroxide) when the first composition and the second compositions are combined, and in some embodiments the concentration is such that the second reactant (peroxide) is in excess with respect to the first reactant (thiourea) when the first composition and the second compositions are combined.

### Peroxide:

One of the reactants that affords FSA is a peroxide, which is used as a source of reactive oxygen. One optional and the simplest peroxide is hydrogen peroxide, or Hydrogen dioxide (H₂O₂). The problem that occurs when using hydrogen peroxide in water (e.g., 50% aqueous solution of H₂O₂ in water) is its stability over time. H₂O₂ tends to decompose very slowly, but even small amount of gas (O₂) in the printheads jetting chambers may cause loss of nozzles, thus causing poor quality, non-uniform print of the delivery composition, and uneven formation of the dye discharge reagent on the surface of the substrate, causing undesired shaded background to the printed image.

Another source of reactive oxygen that are water soluble could be oxidizing salts such as potassium permanganate (the color strong red/violet coloration is problematic) or potassium persulfate and other, but in these salts the oxygen is not so readily available as in peroxides reagents.

Ceric ammonium nitrate (CAN; (NH₄)₂Ce(NO₃)₆) is an optional oxidizing agent which is also contemplated within the scope of some embodiments in the present invention.

According to some embodiments of the present invention, the inkjet printhead used for applying a peroxide, e.g., H₂O₂, is applied using an ink circulation printhead, which can overcome the problem of bubble blockage of nozzles, as discussed hereinabove. Additional information on ink circulation printhead for inkjet applications can be found in the publically accessible literature of the art.

Alternatively, to overcome this problem of peroxide stability, stabilized peroxides can be used, such that decompose and supply the reactive oxygen only when exposed to high temperatures, e.g., of 100° C. and higher. Stabilized peroxides are commercially available in various grades, and typically contain H₂O₂ and chelants/sequestrants which minimize the peroxide decomposition under normal storage and handling conditions. The types of H₂O₂ stabilizers vary between producers and product grades, and generally include:
Colloidal stannate.
Sodium pyrophosphate.
Colloidal silicate (metal sequester that minimizes H₂O₂ decomposition maintaining the bleaching ability of H₂O₂ in alkali media).
Organophosphonates (e.g., Monsanto's Dequest products).
Nitrate (for pH adjustment and corrosion inhibition).
Phosphoric or phosphonic acid (for pH adjustment).

Stabilized peroxides are also provided in, for example, U.S. Pat. Nos. 3,037,622, 3,053,632 4,070,442, 4,132,762, 4,133,869, 4,140,772, 4,155,738, 4,239,643, 4,294,575, 4,304,762, 4,350,681, 4,744,968, 4,895,875, 4,915,781 and 4,981,662, all of which are incorporated herein by reference.

Alternatively or additionally, organic peroxides can be used as a source of reactive oxygen. For example, peroxyesters, dialkyl and diaryl peroxides are of special interest as they are stable and easy to handle compared to peroxyacids and alkyl hydroxy-peroxides. Dialkyl peroxides and peroxyesters, can be turned into water-soluble peroxide salts, such as the commercially available peroxybenzoate converted into water-soluble 4-[[(tert-Butyldioxy)carbonyl]benzyl]triethylammonium chloride [Lu, M. Y. et al., J. Org. Chem., 1995, 60(16), pp 5341-5345].

According to some embodiments of the present invention, the peroxide is selected from the group consisting of hydrogen peroxide, peroxymonosulfuric acid, a peroxomonosulfate salt, a peroxydisulfate salt, an organic peroxy acid, an organic peroxide, an organic hydroperoxide, a perester and a transition metal peroxide.

According to some embodiments of the present invention, the concentration of the reactive species of the peroxide in the second composition ranges from 5-50% by weight of the first composition (0.15M-1.5M). In some embodiments, the concentration of the reactive species of the peroxide in the second composition ranges from 10-20% by weight of the first composition (0.3M-0.6M). It is noted that some peroxides are available and sored as aqueous solutions, typically referred to in terms of the percent of the peroxide in the medium (water), e.g., 50% H₂O₂ in water; in such cases the value of the concentration of the peroxide in the second composition refers to the final/total peroxide content therein. In some embodiments, the concentration of the peroxide in the second composition is determined according to the concentration of the thiourea in the first composition so as to obtain a predetermined molar ratio therebetween.

For example, the molar ratio of the first reactant to the second reactant can be 1:0.2, 1:0.3, 1:0.4, 1:0.5, 1:0.6, 1:0.7, 1:0.8, 1:0.9, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9 or 1:3.

### Other Features and Components of the Dye Discharge Composition:

As presented hereinabove, the combined composition resulting from contacting the first and second compositions (the delivery compositions), according to some embodiments of the present invention, is the reaction mixture where the reagent is being formed in situ, as well as the composition that contains the reagent once it is formed; thus, prior to the completion of the chemical reaction and thereafter, the combined composition comprises essentially the constituents of the delivery compositions apart for the reactants that have been used up in the chemical reaction and any byproducts of the reaction.

In the case of the dye discharge reagent, FSA, the dye discharge composition comprises the first composition and the second composition, wherein the first composition is an aqueous solution of thiourea and the second composition is an aqueous solution of a peroxide. Accordingly, there is provided an inkjet dye discharge composition, comprising a first composition and a second composition, wherein the first composition is an aqueous solution of thiourea and the second composition is an aqueous solution of a peroxide.

According to some embodiments of the present invention, the carrier of the first and second compositions (the delivery compositions) is an aqueous carrier. In some embodiments, the carrier is water (e.g., deionized water). It is understood that the carrier of the dye discharge composition (the combined composition) is also an aqueous carrier.

The dye discharge composition may include additional ingredients, such as for example, surface active agents, surface tension modifying agents, viscosity modifying agents and humectants, as these are known in the art, and exemplified in the following Examples section, and further include optional pH adjusting agents, buffers, binders, film-forming agents, polymerization catalysts, crosslinking agents, softeners/plasticizers, thickeners, anticorrosion agents, anti-foaming agents (defoamers), and any combination thereof. It is noted herein that some agents may serve more than one purpose; for example, a viscosity modifying agent may also serve as a humectant and/or a surface tension modifying agent and/or a surface active agent and, the likes.

Unless mentioned otherwise, the delivery compositions do not contain a colorant and are thus substantially transparent and colorless and intended to leave a bleached-out area on the substrate but not to change the original color to the substrate's material.

When bleaching a dyed substrate, the dye discharged area of the substrate may lose all the dye coloring and exhibit the original color of the substrate material. For example, if the substrate is a dyed cotton fabric, after effecting a bleaching reaction using the dye discharge composition presented herein, the dye discharge area may become yellow, pale yellowish to beige or grayish yellow color. According to embodiments of the present invention, the dye discharge composition may be used to augment and change the original color of the substrate.

Hence, in some embodiments of the present invention, any of the delivery compositions presented herein may further include a colorant or a dye which is impervious to the bleaching effect of the dye discharge reagent, allowing the dye discharge process to impart a pre-selected color to the bleached areas of the substrate. In some of these embodiments, the delivery compositions presented herein may contains a discharge-impervious colorant or dye, such as described, for example, in U.S. Pat. Nos. 4,623,476, 4,474,677, 4,554,091, 4,271,030, 4,464,281, 4,714,562 and 5,089,162.

In some embodiments, any of the delivery compositions presented herein may further include a dispersed colorant/pigment, and in some embodiment may include a colorant/pigment which is dispersed in a film-forming binder and/or an adhesion promoting agent that subsequently forms the printed image on the substrate, as these are known in the art.

In some embodiments the pH of the dye discharge composition (the combined composition) is set to be acidic (pH ranging from, e.g., 2-6). It is noted herein that the pH of the dye discharge composition may be adjusted by adjusting any one of the first and second compositions using an acid, a base, a buffer agent and/or a combination thereof in one or both the delivery compositions. It is further noted that an acidic dye discharge composition, or any one of the delivery compositions if adjusted to be acidic, can also be utilized for immobilizing a colored ink composition, when used in the context of a multi-part ink formulation, designed for printing an image on a colored substrate, as further discussed hereinbelow.

The dye discharge composition may further include agents for effecting immobilization of another ink composition, as discussed hereinbelow. Such dye discharge composition that includes agents for effecting immobilization of another ink composition is useful in the context of a multi-part ink composition as discussed hereinbelow. However, it is noted herein that any of the dye discharge compositions presented herein may be used in the context of any inkjet formulation, as well as one which is not designed to effect or undergo immobilization.

### Kits for Forming In Situ Dye Discharge Reagents:

Any of the delivery compositions presented herein, can be provided to a user in the form of a kit. The term “kit”, as used herein, refers to a single package containing any collection of items or components needed for a specific purpose, especially for use by a user or an operator.

According to an aspect of some embodiments of the present invention, there is provided a kit that includes a packaging material, a first composition and a second composition, wherein the first composition is an aqueous solution of thiourea and the second composition is an aqueous solution of a peroxide packaged therein.

According to some embodiments of the present invention, the kit is in a form wherein some of the ingredients of any one of the compositions presented herein, are packaged individually (separately) within the kit. According to some embodiments of the present invention, the kit may include one or more of the compositions in a ready-for-use form, packaged together within the kit. According to some embodiments of the present invention, the kit contains at least two separate containers for each of the delivery compositions, and can be used for loading two separate inkjet printheads with their contents in preparation for a dye discharge inkjet printing process. In some embodiments, the packaging material is identified in print for use in discharging a dye in a dyed textile substrate.

The kit may further include instructions for the user that includes the following directives:

Load the first composition to a first printhead;

Load the second composition to a second printhead; and

Contact the first composition with the second composition by inkjet printing the compositions on a substrate.

The kit may further include other inkjet composition, such as translucent colored compositions and/or opaque white or colored compositions packaged individually within the kit.

### A Dye Discharged Product:

According to an aspect of some embodiments of the present invention, there is provided a dyed substrate, such as a textile substrate, having at least a portion thereof dyed discharged using a dye discharge reagent which has been formed in situ thereon, as presented herein.

Accordingly there is provided a dyed textile substrate, wherein at least a portion thereof is dyed discharged using the method of dye discharging a substrate as presented herein, or using the inkjet dye discharge composition presented herein.

A dyed textile substrate having an area thereof discharged from the dye using any of the processes, methods or compositions presented herein, may have remnants of one of the reactants and/or may have a dye discharge pattern typical to inkjet, such as small dots of bleached-out surface near the border of discharged and non-discharged sections.

### Printing Color Images on Dye Discharged Substrates:

Any of the processes for in situ preparation of a dye discharge reagent, and any of the methods for dye discharging a dyed substrate, and any of the dye discharge compositions presented herein, is useful also for printing color images on surfaces of dyed substrates as defined hereinabove. Such dye discharge treatment is useful for obtaining a full spectrum of vivid colors in the printed colored image regardless the original color of the dyed substrate.

According to some embodiments of the present invention, the dye discharge reagent which is produced in situ as described herein, can be used in combination with any colored composition that includes a translucent and/or opaque colorants. In such embodiments, an in situ formation of the dye discharge reagent, as presented herein, is effected on the area(s) of the substrate which are predestinated for printing a color image thereon. This dye discharge reagent formation is followed, optionally closely, and optionally almost concomitantly, by applying colored ink compositions on the (optionally still wet) dye discharged areas of the substrate, thereby forming a colored image or design on the colored (dyed) substrate. Such combination could be formed in the context of a multi-part ink formulation, which formed on the surface of the substrate when the first composition, the second composition and one or more colored ink compositions, and possibly other compositions, converge on at least a portion of the surface of the substrate.

### Multi-Part Ink Formulation:

According to some embodiments of the present invention, any of the delivery compositions that form the dye discharge composition presented herein, may form a part (e.g., a discharge part) of a multi-part inkjet formulation which further includes a translucent colored composition (e.g., a colored part), or may be combined with a translucent colored composition for forming an ink formulation (which is used for printing an image).

Since the bleaching process may leave a residual tint or color in the dye discharge areas of the substrate, these areas may require a coat of an opaque white colorant so as to serve as an underbase for the translucent colored composition, thereby allowing the printed image to exhibit a full and vivid spectrum of colors which are obtained when basic translucent CMYK or RGB colors are printed together. Hence, according to some embodiments of the present invention, the delivery compositions forming the dye discharge composition presented herein, and translucent colored compositions as described herein, are used in combination with an opaque underbase composition. According to some embodiments, the opaque underbase composition comprises an opaque white colorant, a dispersing agent and a carrier.

According to other embodiments of the present invention, the delivery compositions presented herein may form a part of a multi-part inkjet formulation which includes an opaque underbase composition for obtaining a desired background for a printed image (e.g., a underbase part), and further includes a translucent colored composition for forming the image.

In the context of embodiments of the present invention, the multi-part ink formulation is obtainable utilizing the delivery compositions presented herein. The ink formulation is obtainable, according to some embodiments, by contacting the delivery compositions on portions of the substrate, and printing a translucent colored composition on these portions of the substrate. According to some embodiments, the ink formulation is also obtainable by printing an opaque underbase composition on these portions of the substrate and further printing a translucent colored composition on these portions of the substrate.

In some embodiments, the color composition is suitable for use in inkjet printing. In some embodiments, the color composition is a translucent colored composition. In some embodiments, the translucent colored composition may be the same or resemble, at least in some characteristics and ingredients, many standard and common inkjet compositions.

According to embodiments of the present invention, the translucent colored composition comprises a colorant, a dispersing agent and a carrier. According to embodiments of the present invention, the dispersed colorant/pigment, typically a cyan, magenta, yellow and black colorant (CMYK), or red, green and blue colorant (RBG), is impervious to the reducing agent of the dye discharge composition. According to some embodiments of the present invention, the carrier is an aqueous carrier. In some embodiments, the carrier is water.

### The Dye Discharge Composition as an Immobilization Composition:

In order to obtain a sharp and vivid image by inkjet in the form of a film attached to a surface of a substrate, the film should be made from fine and distinct points of colors, corresponding to pixels of a digital image or the grain of a photographic paper coated with light-sensitive chemicals. These distinct points of colors stem from very fine droplets of the translucent colored composition which are jetted onto the substrate during the printing process, and the finer the droplets remain on the substrate after ejection, the finer the image would be. The droplets will remain fine if feathering, bleeding and smearing can be limited.

One way to afford a sharp image film on the substrate is to “freeze” or immobilize the ink droplets on contact with the substrate. Thus, a chemical and/or physical change takes effect in the ink composition upon contacting thereof with the substrate, and this chemical and/or physical change is effected by contacting agents in various compositions, which are designed to afford the immobilization of the inkjet droplets on the substrate, which will eventually lead to better and sharper images.

While contemplating embodiments of the present invention, it has been contemplated that any of the delivery compositions or the combined (dye discharge) composition presented herein may also serve to effect immobilization of the translucent colored composition, if the latter comprises substances that congeals or coagulates upon contact with the dye discharge composition. Hence, according to some embodiments of the present invention, the translucent colored composition and the dye discharge compositions are formulated such that the colored composition congeals or coagulates upon contact with the dye discharge composition, thereby forming an immobilized plurality of ink dots on the dye discharged area of the dyed substrate, thereby collectively form an image thereon.

The term “immobilization”, as used in the context of embodiments of the present invention, refers to the act of restriction or substantial limitation of flowability of a liquid, namely substantial reduction of the capability of a liquid to move by flow. For example, immobilization of a liquid can be effected by congelation of the liquid or solutes therein. Immobilization of droplets of liquid ink can be achieved, for example, by elevating the viscosity of the liquid ink composition such that the droplets are restricted from flowing once in contact with the substrate. As used herein, the term “immobilization” is not meant to include final polymerization and print fixation by crosslinking and curing reactions.

Quantitatively, “immobilization” in the context of embodiment of the present invention is defined as elevating the viscosity of the color-bearing parts of the ink composition by 10-folds, 50-folds, 100-folds, 500-folds 1000-folds or 2000-folds and more. For example, when a given color-bearing part is characterized by having a viscosity of 10-13 cp, it is defined as immobilized when its viscosity is elevated to about 2000 cp or higher as a result of congelation.

Hence the chemical and/or physical change, which effects the aforementioned immobilization droplets of liquid ink, according to some embodiments of the present invention, is congelation. The term “congelation”, as used herein, is synonymous to the terms “coagulation”, “thickening” or “gelation”, and refer to the sharp decrease in fluidity of a formerly fluid liquid. Congelation can be effected also by sedimentation, precipitation, partial solidification and partial polymerization of soluble constituents in the composition. The term “coagulation”, as used herein, refers to the destabilization of suspended colloidal or emulsified substances. The term “flocculation”, as used herein, refers to the bridging between particles by a polymer chain, causing them to form flocs or larger aggregates that may sediment or precipitate.

### Various Additives of Ink Compositions:

Following are descriptions and examples of various ingredients of the various compositions presented herein.

Exemplary film-forming agents include, without limitation, non-ionic water-emulsifiable resins such as acrylic polymers and copolymers, alkyl-acrylic polymers and copolymers, acrylic-styrene copolymers, polyurethanes, polyethers, polyesters, polyacrylates and some combinations thereof.

According to some embodiments of the present invention, the film-forming agent is a self-crosslinking alkyl-acrylic copolymer, and according to some embodiments, the self-crosslinking alkyl-acrylic copolymer is an ethyl-acrylic/butyl-acrylic copolymer. Some acrylic-based polymers and copolymers are emulsifiable self-crosslinking polymers which are used in the garment industry to bestow physical and chemical resistance to the cloth, knitted, woven or non-woven, against physical wear and tear due to frequent use and repeated washing, as well as against alcohol, organic solvents and water.

When selected to have a low Tg, according to embodiments of the present invention, commercially available suitable film-forming agents, which may also serve as pigment dispersants in a relevant composition, include without limitation, TEXICRYL™ 13-216 (Tg −14° C.), TEXICRYL™ 13-290 (Tg −30° C.), TEXICRYL™ 13-297 (Tg −9° C.) and TEXICRYL™ 13-326 (Tg −25° C.) which are commercially available from Scott Bader Ltd., and APPRETAN™ E 2100 (Tg −30° C.), JONCRYL™ 617 (Tg +7° C.), APPRETAN™ E 6200 (Tg −20° C.) and APPRETAN™ E 4250 (Tg −15° C.) which are commercially available from Clariant.

Other non-limiting examples of commercially available film-forming agent ACRYSOL™ series, commercially available from Rhome and Hass Ltd., and ACRONAL™ series, commercially available from BASF Inc.

Other binders, dispersants and adhesion promoters which are useful in the context of an elastomeric film-forming agent according to embodiments of the present invention include, without limitation, commercially available and widely used families of products, known under names such as Alkydal™, Desmodur™ and Desmophen™ (from Bayer); Beckopox™, Macrynal™, Maprenal™, Viacryl™ and Vialkyd™ (from Vianova Resins); Cythane™ (from Cytec); Dynapol™ and Vestanat™ (from Hiils); Johncryl™ (from Johnson); K-Flex™ (from King Industries); Synocure™ and Synolac™ (from Cray Valley); Synthalat™ (from Synthopol); Tolonate™ (from Rhone Poulenc); Uracron™ and Uralac™ (from DSM); Worleecryl™ and Worleekyd™ (from Worlee) and the likes.

It is noted that any exemplary ingredient disclosed herein is given for exemplifying purposes only, and should not be regarded as limiting to that particular ingredient or commercially available product, but rather regarded as a representative member of a wider group of alternatives, all of which are meant to be encompassed in the context of other embodiments of the present invention.

Additional optional ingredients in the various compositions presented herein include surface active agents and viscosity modifying agents.

Exemplary surface active agents include, without limitation, polyether modified poly dimethyl siloxanes, polymethylalkylsiloxane, polyester hydroxyl modified poly-dimethyl-siloxane, a fluorocarbon/hydrocarbon anionic surfactant, a polyacrylic copolymer and any combination thereof.

Exemplary viscosity modifying agents include, without limitation, associative thickeners, Newtonian rheology additives, glycols, polyethyleneglycol, propylene glycol, triethylene glycol, diethylene glycol, glycerin, high molecular weight alcohols, carbowaxes, polyvinyl alcohols, polyvinyl pyrrolidones, and any combination thereof.

Exemplary anticorrosion agents include, without limitation, tolyltriazole (methylbenzotriazole, benzotriazole, and combinations thereof.

It is expected that during the life of a patent maturing from this application many relevant methods, uses and compositions will be developed and the scope of the terms methods, uses, compositions and polymers are intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the phrase “substantially devoid of” a certain substance refers to a composition that is totally devoid of this substance or includes no more than 0.1 weight percent of the substance.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The words “optionally” or “alternatively” are used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Dye Discharge Composition

A dye discharge composition, according to some of embodiments of the present invention, is formulated in two separate compositions, referred to herein as a first composition and a second composition, wherein the first composition comprises thiourea and the second composition comprises a peroxide. The following is a general and a particular compositions listing the ingredients, with amounts given in percent by weight of the total weight of the composition, and relative amounts of the ingredients are given as percent by weight unless stated otherwise.

First Composition (General):

| Thiourea | 10-15% (0.13M-0.2M) |
|---|---|
| Viscosity modifying agent/Humectant | 20-70% |
| Buffering agent | 5-15% |
| Surface active (wetting) agent | 0-2% |
| Defoamer | 0-2% |
| Various other additives | 0-20% |
| Deionized water | QS to 100% |
| pH buffered to | 8-10 |

Second Composition (General):

| A peroxide (reactive species) | 10-20% (0.3M-0.2M) |
|---|---|
| Peroxide stabilizing agent | 0-20% |
| Viscosity modifying agent/Humectant | 20-70% |
| Surface active (wetting) agent | 0-2% |
| Defoamer | 0-2% |
| Various other additives | 0-20% |
| Deionized water | QS to 100% |
| pH buffered to | 4-7 |

The following exemplary inkjet dye discharge composition, formulated in two exemplary first and second compositions, according to some of embodiments of the present invention, was prepared as follows:

First Composition (Specific Embodiment):

| Thiourea | 13% (0.172M) |
|---|---|
| Propylene glycol (PG) | 55% |
| Ethylene glycol (MEG) | 5% |
| Ammonium lactate | 9% |
| Deionized water | QS to 100% |
| pH buffered to | 8.5-9.5 |

Second Composition (Specific Embodiment):

| H₂O₂ (50% aq. stock) | 30% (0.44M) |
|---|---|
| Propylene Glycol | 70% |
| Deionized water | QS to 100% |
| pH buffered to | 6.0-7.0 |

The two compositions were used in the following example to form a dye discharge composition in situ on the surface of a darkly dyed piece of fabric.

### Example 2

The following is an exemplary process using a dye discharge composition, according to some embodiments of the present invention. It should be noted that this exemplary process is applicable for use in combination with other transparent or opaque colored and opaque white ink compositions, some of which are presented herein.

The exemplary printing process, according to embodiments of the present invention, was performed as follows:

A darkly brown-dyed cotton substrate was placed in a printing machine equipped with a plurality of printheads (Kornit™ “Drop-On-Demand” Thunder™ inkjet printing machine.

Each of the first and second compositions was loaded into a separate printhead, where second composition comprising the peroxide, was loaded into an ink circulation printhead. It is noted that an ink circulation printhead is advantageous in the case of using H₂O₂ as a peroxide since it prevents clogging the printhead by bubbles of oxygen which may appear in the reservoir and tubing due to spontaneous decomposition of hydrogen peroxide.

An image in the form of a rectangular 20×20 matrix was pre-configured on a computer. The programmable digital information of the image was fed into the computerized controller of the printing machine, and the plurality of printheads were driven over the substrate. Using the printing machine, an exemplary the dye discharge composition, comprising a first composition (thiourea) and a second composition (peroxide), according to the example presented in Example 1 hereinabove, was applied at varying quantities of each of the compositions on the surface of the brown-dyed cotton fabric in grid made of a series of printed squares (a matrix). The Y axis in the printed matrix represent the relative amount of the first composition comprising thiourea in the dye discharge composition (the mixture of the first and second compositions as formed on the substrate), and the X axis represents the relative amount of the second composition comprising hydrogen peroxide in the dye discharge composition. The axes values represent the fraction of the output capacity of the respective nozzle in percent, going from 20% to 77% of each of the components, wherein each grid step represent a 3% increase in the output capacity of the respective nozzle of each composition, and the total of both components along the minor diagonal (from the bottom left corner to the top right corner of the matrix) represents combined compositions of equal amounts. As the reaction for producing FSA requires stoichiometric amount of thiourea and H₂O₂ (1:2 molar amount ratio, respectively), the main diagonal of the matrix (from bottom-left to upper-right corners) represents increasing amounts of the dye discharge composition, the area above the main diagonal represents dye discharge composition with excess of thiourea, and the area below the main diagonal represents dye discharge composition with excess of H₂O₂. The printed image can serve as a Discharge/Devoré Selection Chart for a particular substrate, which can be used to select the relative and absolute amounts of each of the first and second compositions that will achieve a desired effect, ranging from strong dye discharge (bleaching) to moderate dye discharge, and from moderate fabric burnout (Devoré) to complete fabric etching.

The printheads containing first and the second compositions applied their contents concurrently strip-wise according to the digitized matrix design. Subsequently, the substrate was moved into a conveyer oven operated at 140-150° C. for 1-10 minutes.

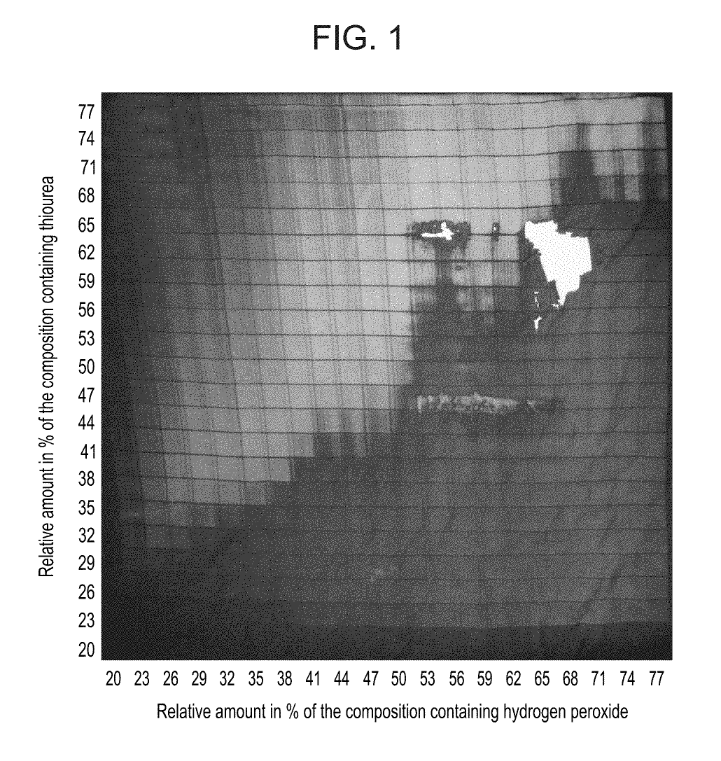

FIG. 1 is a photograph of an exemplary Discharge/Devoré Selection Chart, which was formed on a sample of a brown-dyed cotton fabric sample on which a dye-discharge composition, according to some embodiments of the present invention, was applied, showing the various dye-discharge and/or fabric burnout (devoré) levels resulting for the application of various amounts of the two components comprising the dye discharge composition.

As can be seen in FIG. 1, the matrix cells where the amount of H₂O₂ exceeded the amount of thiourea, the fabric was burned-out by the acidic oxidant, while the cells where the amounts of H₂O₂ and thiourea were at or near equal, the fabric remained intact and the dye was discharged, leaving a yellowish cloth. As can further be seen in FIG. 1, the matric cells where the amount of thiourea exceeded the amount of H₂O₂, the dye on the fabric remained visible, indicating that the dye discharge composition in these cells contains less than the amount of the dye discharge agent than required to effect bleaching of the fabric.

It is noted that the color of the resulting dye-discharged square areas (the grid elements of the matrix) can be determined in terms of Lab* values, and the resulting Lab* values can serve as a quantified “Discharge/Devoré Selection Chart”.

### Example 3

The following is a description of a printing process wherein additional colors are printed over the fabric which has been treated with the dye discharge composition according to some embodiments of the present invention.

Hence, optionally, other separate printheads may be loaded with other ink compositions, such as:

A translucent cyan colored composition;

A translucent magenta colored composition;

A translucent yellow colored composition;

A translucent black colored composition; and

An opaque white underbase composition.

Optionally additional colored ink compositions such as green, red, light colors and others can be added in additional separate printheads so as to extend the range of color gamut/spectrum.

Once the portion of the substrate corresponding to the silhouette of the image is applied with the dye discharge composition (the combined solutions; see Examples 1 and 2), and prior to applying heat to the printed substrate, the printheads containing the opaque white composition and the printheads containing the CMYK compositions, may apply a mixture of opaque and/or translucent ink compositions over the same portion of the area where the dye has been discharged from the substrate.

It is noted that this process may be effected without applying an underbase layer of an opaque white colorant, particularly in substrates which exhibit undergoes a dye discharge reaction which affords a bright bleached-out material, or when the specific requirements allow the design to be printed over the bleaches area without further whitening thereof.

Optional Colored Composition:

A typical translucent colored composition, according to some embodiments of the present invention, generally formulated using the following ingredients, with amounts given in percent by weight of the total weight of the composition:

| | |
|---|---|
| Pigment | 1.5-5% |
| Dispersant | 1-9% |
| Surfactants | 0.1-2.0% |
| Film-forming agent | 10-25% |
| Viscosity modifying agent/Humectant | 10-40% |
| Buffer (basic/amine) | 0.1-0.4% |
| Deionized water | QS to 100% |
| pH Buffering | higher than 7 |

The following exemplary translucent colored composition, according to some embodiments of the present invention, was prepared:

| | |
|---|---|
| Pigment (C,M,Y or K) | 2-4% |
| Carboxylated polymer (Dispersant) | 5% |
| Surfactant Polyether dimethyl siloxane | 2% |
| Glycols/glycerin | 40% |
| Emulsified acrylic polymer (40-50% solid) | 30% |
| Carbodiimide crosslinker | 3% |
| Amine (buffering) | 0.1-0.4% |
| Deionized water | QS to 100% |
| pH Buffering | 7.5 |

Optional Opaque Underbase Composition:

A typical opaque underbase composition, according to some embodiments of the present invention, is generally formulated using the following ingredients, with amounts given in percent by weight of the total weight of the composition:

| | |
|---|---|
| Opaque white pigment | 8-12% |
| Dispersant | 4% |
| Surfactant | 0.3-0.6% |
| Defomer | 0.3-0.6% |
| Film-forming agent emulsion | 40-50% (40-50% solids) |
| Crosslinkers | 1-3% |
| Humectant | 35-50% |
| Buffer (basic/amine) | 0.1-0.4% |
| Deionized water | QS to 100% |
| pH Buffering | higher than 7 |

The following exemplary opaque underbase composition, according to some embodiments of the present invention, was prepared:

| | |
|---|---|
| Titania | 10% |
| High MW polyacrylate dispersant | 3.8% |
| Polyether dimethyl siloxane | 0.4% |
| Acrylate sodium salt | 1.5% |
| Glycols | 25% |
| Acrylic emulsion | 38% |
| Carbodiimide crosslinker | 3% |
| Tertiary Amine | 0.2% |
| Deionized water | QS to 100% |
| pH Buffering | 7.5 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. A method of discharging a dye in a dyed substrate, comprising contacting at least a portion of said substrate with a first composition and a second composition, wherein said first composition comprises a first reactant and said second composition comprises a second reactant, said first reactant and said second reactant undergo a chemical reaction to form a dye discharge reagent in situ on the substrate, thereby discharging the dye in the substrate with said dye discharge reagent, and wherein said contacting is afforded by separately applying said first composition and said second composition on said portion.

3. (canceled)

4. The method of claim 2, wherein said first reactant and said second reactant are applied at a stoichiometric ratio.

5. (canceled)

6. The method of claim 2, wherein each of said first composition and said second composition is individually applied on said portion of the substrate by a separate inkjet printhead.

7. (canceled)

8. The method of claim 2, wherein said applying is effected concertedly, concomitantly or sequentially.

9. (canceled)

10. The method of claim 2, further comprising heating and/or irradiating at least said portion of the substrate subsequent to said contacting.

11-12. (canceled)

13. The method of claim 2, wherein said first reactant is thiourea and said first composition is an aqueous solution of said thiourea.

14. The method of claim 13, wherein said second reactant is a peroxide and said second composition is an aqueous solution of said peroxide.

15. The method of claim 14, wherein said dye discharge reagent formed in situ is formamidine sulfinic acid (FSA).

16. (canceled)

17. The method of claim 15, wherein a concentration of said thiourea in said first composition ranges from 5-25%.

18. The method of claim 15, wherein said peroxide is selected from the group consisting of hydrogen peroxide, peroxymonosulfuric acid, a peroxomonosulfate salt, a peroxydisulfate salt, an organic peroxy acid, an organic peroxide, an organic hydroperoxide, a perester and a transition metal peroxide.

19. The method of claim 18, wherein a concentration of said peroxide in said second composition ranges from 5-50%.

20-22. (canceled)

23. A kit comprising a packaging material and the inkjet dye discharge composition that comprises a first composition and a second composition, wherein said first composition is an aqueous solution of thiourea and said second composition is an aqueous solution of a peroxide.

24. The kit of claim 23, being identified for use in discharging a dye in a dyed textile substrate.

25. The kit of claim 23, being identified for use in a method of inkjet printing an image on at least a portion of a dyed textile substrate.

26. The kit of claim 23, wherein said first composition and said second composition are packaged individually within the kit.

27. The kit of claim 26, further comprising instructions that comprises:
loading said first composition to a first printhead;
loading said second composition to a second printhead; and
contacting said first composition with said second composition by inkjet printing said first composition and said second composition on a substrate.

28. The kit of claim 25, further comprising a translucent colored composition packaged individually within the kit.

29. The kit of claim 25, further comprising an opaque underbase composition packaged individually within the kit.

30-31. (canceled)

32. A process of inkjet printing an image on at least a portion of a dyed textile substrate, the process comprising digitally applying onto at least a portion of a substrate a inkjet dye discharge composition that comprises a first composition and a second composition, and digitally applying substantially over said portion a translucent colored composition, thereby forming the image, wherein said first composition is an aqueous solution of thiourea and said second composition is an aqueous solution of a peroxide.

33. The process of claim 32, further comprising, prior to applying said translucent colored composition, digitally applying an opaque underbase composition substantially over said portion.

34. The process of claim 32, wherein applying said dye discharge composition and applying said translucent colored composition, and/or applying said dye discharge composition and applying said opaque underbase composition, if present, is effected substantially concurrently.

35. The process of claim 32, subsequent to said applying, heating the substrate.
